(19) United States
(12) Patent Application Publication
(10) Pub. No.: US 2008/0014176 A1
(43) Pub. Date: Jan. 17, 2008

(54) METHOD OF INDUCING TOLERANCE TO BETAAP 1-42 AND MYELIN BASIC PROTEIN

(76) Inventors: Thomas M. Di Mauro, Southboro, MA (US); Chantal Holy, Raynham, MA (US); Mohamed Attawia, Canton, MA (US); Sean Lilienfeld, Sharon, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/457,822

(22) Filed: Jul. 17, 2006

(51) Int. Cl. A61K 35/14; C12N 5/08; C12N 15/00

(52) U.S. Cl. 424/93.7; 435/372; 435/440

(57) ABSTRACT

UVB irradiation of white blood cells in order to induce tolerance to antigenic βAP 1-42. To treat Alzheimer's Disease in a patient, irradiate autologous white blood cells with UVB light to cause tolerance therein. Combine the tolerized cells with βAP 1-42 to form a mixture. To treat stroke or multiple sclerosis in a patient, irradiate autologous white blood cells with UVB light to cause tolerance therein. Combine the tolerized cells with myelin basic protein to form a mixture.

### BACKGROUND OF THE INVENTION

In Alzheimer's Disease (AD), the cleavage of beta amyloid protein precursor from the intracellular membrane often produces a protein βAP 1-42 which is incompletely removed by normal clearance processes. Over time, this protein is deposited as a beta amyloid protein Aβ plaque within brain tissue, leading to the local destruction of neurons. The Aβ plaque deposition is also believed to provoke an inflammatory response by microglia and macrophages, which recognize the plaque as a foreign body. These cells are believed to respond to the plaque deposition by releasing pro-inflammatory cytokines and reactive oxygen species (ROS). Although the inflammatory response may be provoked in an effort to clear the brain tissue of the detrimental plaque, it is now believed that this inflammation also injures local neuronal tissue, thereby exacerbating AD.

In most AD cases, the progression of AD begins in the hippocampus, wherein the patient suffers a loss of short term memory. From the hippocampus, the disease spreads to the amydgala, and then proceeds anteriorly to the prefrontal cortex. Since the prefrontal cortex controls problem-solving, a person suffering from AD begins to lose their ability to learn when the disease affects the prefrontal cortex. In general, impairment of the prefrontal cortex begins to appear a few years after loss of short-term memory.

Because of the role played in AD by inflammation, anti-inflammatory compounds have been identified as candidates for treating Alzheimer's Disease. However, the delivery of these compounds has generally been through an oral route, and the systemic side effects associated with long term use of these compounds are often undesirable. Some investigators have proposed implanting an effective amount of NGF in a sustained release device for treating Alzheimer's Disease. However, NGF simply helps restore damaged neurons—it does little to stop the damage from occurring.

### SUMMARY OF THE INVENTION

The present invention relates to the UVB irradiation of white blood cells in order to induce tolerance to antigenic βAP 1-42.

In one embodiment of the present invention, UVB-irradiated, autologous macrophages and lymphocytes are combined ex vivo with βAP 1-42 and then injected into the patient near in the vicinity of a lymph node.

The UVB irradiation of the macrophages will change the character of the antigen presenting function of the macrophages from a pro-inflammatory to an anti-inflammatory character. Accordingly, when the tolerogenic macrophage engulfs a βAP 1-42 and presents it to B cells in the lymph node, the resulting immune response will be that of a Th2 anti-inflammatory response.

Likewise, UVB irradiation of the lymphocytes will induce a release of anti-inflammatory IL-10 therefrom. The increase in IL-10 in the region surrounding the macrophages will further polarize the immune response to an anti-inflammatory Th2 response.

Support for the concept of providing UVB irradiation of WBCs to induce tolerance to antigens is found in the literature, wherein it has been reported that UVB irradiation of white blood cells (WBCs) causes immune tolerance to the subsequent implantation of allogenic transplants. Kao, Blood, 88(11), Dec. 1, 1996, 4375-82 and Xia, Transfusion, 45, February 2005 181-188.

Therefore, in accordance with the present invention, there is provided a method of treating AD, comprising the steps of:
a) irradiating autologous macrophages with UVB light to cause tolerance therein, and
b) combining the tolerized macrophages with βAP 1-42.

The UVB irradiated cells should behave in a very tolerogenic, anti-inflammatory manner and produce systemic tolerance to the antigens they encounter. In particular, the antigen-presenting function of macrophages will become tolerogenic, while the lymphocytes should begin to emit IL-10.

In preferred embodiments, this procedure once a week for about 4 weeks, thereby provoking a desirable, long-lasting tolerance of βAP 1-42.

### DESCRIPTION OF THE FIGURES

FIG. 1 is a cross-section of a syringe of the present invention having a UVB-producing unit attached thereto.

FIG. 2 is a cross-section of a centrifugation container filled with whole blood.

FIG. 3 is a cross-section of a centrifugation container filled with centrifuged blood.

FIG. 4 is a side view of a syringe filled with βAP 1-42 having a needle inserted into the container of FIG. 3.

FIG. 5 is a side view of the syringe of FIG. 3 having a UVB-producing unit attached thereto.

FIG. 6 is a cross-section of a syringe of the present invention injecting tolerized white blood cells into the vicinity of a lymph node.

### DETAILED DESCRIPTION OF THE INVENTION

In some embodiments, the βAP 1-42 is obtained as a recombinant protein. In other embodiments, the βAP 1-42 protein is obtained autologously.

In other embodiments, the βAP 1-42 protein is provided by a non-neuronal organ, such as the thyroid or liver. The literature has reported that antigenic βAP 1-42 fibrils are found not only in neuronal tissue, but in non-neuronal tissue as well. One particular organ that has been repeatedly found to have antigenic βAP 1-42 fibrils is the thyroid.

Therefore, in some embodiments, UVB irradiated WBCs are re-injected back into the patient in the vicinity of the thyroid gland. These tolerized WBCs then engulf βAP 1-42 fibrils present in the thyroid and present this antigen to nearby lymph nodes in a tolerogenic manner.

In other embodiments, the βAP 1-42 protein is provided by the patient's blood. The literature has reported the prescence of significant amounts of βAP 1-42 in the blood stream of AD patients: Lewczuk, Electrophoresis, 2004 Oct. 25 (20) 3336-43.

In other embodiments, the βAP 1-42 protein is provided by the patient's cerebrospinal fluid (CSF). The literature has reported the prescence of significant amounts of βAP 1-42 in the CSF of AD patients: Wiltfang, Electrophoresis 1997 March-April 18(3-4) 527-32; and Klafki, Anal. Biochem. 1996 May 15, 237(1) 24-9.

Therefore, in preferred embodiments, isolation of autologous βAP 1-42 is achieved by a procedure comprising the steps of:
a) drawing a fluid selected from the group consisting of blood or CSF from the patient,
b) combining the fluid with PAGE to form a mixture,
c) applying an electrophoretic voltage across the mixture to isolate the βAP 1-42, and
d) removing the isolated βAP 1-42 from the mixture.

In some preferred embodiments, the autologous βAP 1-42 is obtained by isolation via PAGE electrophoresis. The literature has reported the successful electrophoretic isolation of βAP 1-42 via electrophoresis: Lewczuk, Electrophoresis, 2004 October 25 (20) 3336-43; Wiltfang, Electrophoresis 1997 March-April 18(3-4) 527-32; and Klafki, Anal. Biochem. 1996 May 15, 237(1) 24-9.

Moreover, the present inventors have noted that the PAGE process uses polyacrylamide as a separation gel. The literature has reported that polyacrylamide surface tend to provoke an anti-inflammatory Th2 response, causing white blood cells to emit copious amounts of IL-10.

Therefore, since it is appreciated that the polyacrylamide may be helpful to the present invention by inducing a further polarization of the immune response to a Th2 immune response, in some embodiments, both the autologous βAP 1-42 and the polyacrylamide are combined with the tolerized white blood cells.

In preferred embodiments, WBC tolerance is achieved by a procedure comprising the steps of:
a) drawing blood from the patient suffering from AD,
b) centrifuging the blood to isolate the lymphocytes and macrophages therein,
c) withdrawing the lymphocytes and macrophages from the centrifuged blood,
d) UVB irradiating the lymphocytes and macrophages to cause tolerance.

In preferred embodiments, a physiologic fluid containing viable WBCs is obtained from the patient. Preferably, the physiologic fluid is whole blood. Whole blood contains monocytes and lymphocytes and is easily obtainable from the patient. More preferably, the obtained whole blood is then fractionated by a conventional procedure (such as centrifugation or filtration) to obtain a selected portion of whole blood.

In some embodiments, the selected portion comprises the buffy coat fraction of whole blood. The buffy coat typically comprises about 5-10 vol % of whole blood Utilization of the buffy coat in the present invention is desirable because it contains a concentrated amount of monocytes and lymphocytes. Typically, the cellular concentration in the buffy coat will be on the order of 10-20 fold over whole blood. In some embodiments, a fraction of the buffy coat may be used.

Preferably, white blood cells are selected as the viable cells are the present invention. Because these cells are easily obtained in a concentrated form from the simple centrifugation of a small amount of blood taken from the patient. More preferably, the monocyte fraction of white blood cells is selected as the viable cells of the present invention, as monocytes have been shown to become tolerogenic upon irradiation by UVB light. In other embodiments, the white blood cell fraction is lymphocytes.

In one embodiment, filtration and dewatering of blood is carried out in accordance with U.S. Pat. No. 5,733,545 (Hood) to obtain a buffy coat having about 14×10⁶ monocytes/ml.

In some embodiments, the white blood cells comprise lymphocytes. In others, the white blood cells comprises immature dendritic cells.

Once the WBCs are tolerized and isolated βAP 1-42 is obtained, the two are combined ex vivo and then re-injected back into the patient, preferably in the vicinity of a lymph node that forms microglia.

High levels of aluminum, copper, iron, and zinc have been found in the brains of AD patients. For example, Finefrock, J. Am. Geriatr. Soc., 51, 1143-1148, (2003) reports the following concentrations:

| | Total Amyloid Plaque | AD Neuropil | Control neuropil |
|---|---|---|---|
| Metal | (ug/g) | (ug/g) | (ug/g) |
| Copper | 25 | 19 | 04 |
| Iron | 53 | 39 | 19 |
| Zinc | 69 | 51 | 23 |
| Aluminum | — | — | — |

### It has been hypothesized by Finefrock, supra, that age-related dyshomeostasis and environmental accumulation are responsible for these high metal levels.

Furthermore, it is believed that these heavy metals play a critical role in the precipitation of βAP. It is known that βAP binds to these heavy metals and even has highly specific binding sites for copper. Accordingly, high levels of these heavy metals have been found in βAP plaques. Huang, J. Nutrition, 2000, May 130(5S Supp.) 1488S-92S). It has been reported that zinc may serve a twin role by both initiating amyloid deposition and then being involved in mechanisms attempting to quench oxidative stress and neurotoxicity derived from the amyloid mass. Huang, J. Nutrition, 2000, May 130 (5S Supp.) 1488S-92S, and Finefrock, J. Am. Geriatr. Soc., 51, 1143-1148, (2003)

Since the presence of heavy metals may help determine the epitopic configuration of a βAP 1-42 aggregates, in some embodiments, at least one heavy metal is added to the isolated βAP 1-42 solution in order to mimic the βAP 1-42 precipitation that occurs in the AD brain.

In some embodiments, the patient has a copper (+2) concentration in the βAP 1-42 containing solution of at least 1 μM. In some embodiments, the patient has a copper (+2) concentration in the βAP 1-42 containing solution of at least 10 μM. In some embodiments, the patient has a zinc (+2) concentration in the βAP 1-42 containing solution of at least 1 μM. In some embodiments, the patient has a zinc (+2) concentration in the βAP 1-42 containing solution of at least 10 μM. In some embodiments, the patient has an iron (+3) concentration in the βAP 1-42 containing solution of at least 1 μM. In some embodiments, the patient has an iron (+3) concentration in the βAP 1-42 containing solution of at least 10 μM.

In some embodiments, the UVB light source has a spectral maximum in the range of the between 280 nm and 320 nm. In some embodiments, the light source has a spectral maximum of about 311 nm-312 nm. Preferably, the UVB light source is characterized as a narrowband light source. The literature has reported a lack of carcinogenicity in narrowband UVB light sources in the range of about 312 nm

In some embodiments, the UV light source is situated to irradiate adjacent tissue with between about 0.02 J/cm² and 20 J/cm² energy. Without wishing to be tied to a theory, it is believed that light transmission in this energy range will be sufficient to activate the macrophages and astrocytes of most brain tissue. Shreedhar, J. Immunol., 1998, 160, 3783-9 has reported using a light dose of 0.02 J/cm² in order to activate keratinocytes to produce IL-10. Schmitt, J. Immunology, 2000, 165:3162-7 has reported using a dose of 1.5 J/cm². Rivas, J. Immun, 149, 12, 1992, 3865-71 has reported using a dose of 0.02 J/cm². Therefore, it is believed that irradiating inflamed brain tissue with at least about 0.02 J/cm² of UV radiation will induce the macrophages and microglia therein to produce and emit I1-10. In some embodiments, the light source is situated to produce an energy intensity at the cell surface of between 0.1 watts/cm² and 10 watts/cm². In some embodiments, the light source is situated to produce about 1 milliwatt/cm². This latter value has been reported by Ullrich to effectively irradiate a cell surface in an amount sufficient to produce IL-10.

In some embodiments, the tolerized WBCs and isolated βAP 1-42 are combined ex vivo and then re-injected into the patient in the vicinity of a lymph node. Preferably, the lymph node is one that serves the brain. Preferred lymph nodes include cervical lymph nodes.

In some embodiments, the tolerized WBCs and isolated βAP 1-42 are combined ex vivo and then re-injected into the patient in the vicinity of mucosa-associated lymphoid tissue (MALT). Mucosal administration of certain antigens causes suppressor T-cells to be induced in mucosa-associated lymphoid tissue (MALT). These antigen-specific suppressor T-cells are released in the blood or lymphatic tissue and then migrate to the organ or tissue afflicted by the autoimmune disease (which has a high concentrated of the antigen). Once they have arrived at their intended target, these suppressor T-cells mediate the release of immunosuppressive cytokines such as transforming growth factor β (TGF-β), IL-4 and/or IL-10 and thereby suppress autoimmune attack of the afflicted organ or tissue.

In more detail, the mechanism of bystander suppression is as follows: After a tissue-specific bystander antigen is mucosally administered, it passes to local lymph tissue (such as Peyers Patches in the gut), which contain T cells and B cells. These cells, are in turn in communication with the immune system, including the spleen and lymph nodes. The result is that suppressor (CD8+) T-cells are induced and recruited to the area of autoimmune attack, where they cause the release of TGF-β, IL-4 and IL-10, which can non-specifically downregulate the B-cells as well as the activated CD4+T-cells directed against the mammal's own tissues. Despite the non-specific nature of the activity of these cytokines, the resulting tolerance is specific for the autoimmune disease by virtue of the fact that the antigen is specific for the tissue under attack and suppresses the immune cells that are found at or near the tissue being damaged.

TGF-B is an anti-inflammatory cytokine that helps polarize the immune response towards a Th2 phenotype. IL-4 and IL-10 are also antigen-nonspecific immunoregulatory cytokines. IL-4 in particular enhances Th2 response, i.e., acts on T-cell precursors and causes them to differentiate preferentially into Th2 cells at the expense of Th1 responses. IL-4 also indirectly inhibits Th1 exacerbation. IL-10 is a direct inhibitor of Th1 responses. After orally tolerizing mammals afflicted with autoimmune disease conditions with bystander antigens, increased levels of TGF-β, IL-4 and IL-10 are observed at the locus of autoimmune attack. Chen, Y. et al., Science, 265:1237-1240, 1994.

The action of these cytokines is not specific for the antigen triggering the suppressor cells that release them, even though these suppressor T-cells release these cytokines only when triggered by the mucosally-administered antigen. However, because mucosal tolerization with the antigen only causes the release of these cytokines in the vicinity of autoimmune attack, no systemic immunosuppression ensues. Recruitment of the suppressor T-cells to a locus where cells contributing to the autoimmune destruction are concentrated allows for the release of these suppressive cytokines in the vicinity of the disease-causing cells and suppresses (i.e. shuts down) these cells. The ability of these immunosuppressive cytokines to suppress these “destructive” cells is independent of the antigen for which the destructive cells may be specific.

In some embodiments, the tolerized WBCs and isolated BAβ 1-42 are combined ex vivo and then re-injected into the patient in the vicinity of the nasal-associated lymphoid tissue (NALT)

U.S. Pat. No. 6,645,504 (“Weiner I”) and U.S. Pat. No. 5,935,577 (“Weiner II”) each discloses that certain synergists can be co-administered along with the antigen to enhance the effectiveness of the tolerance-promoting treatment. Particularly, noted is the use of IL-4; IL-10; bacterial lipopolysaccharides; immunoregulatory lipoproteins; and cholera toxin β-chain (CTB). Therefore, in some embodiments, a synergists selected from the group consisting of IL-4; IL-10; bacterial lipopolysaccharides; immunoregulatory lipoproteins; and cholera toxin β-chain (CTB) can be co-administered along with the antigen to enhance the effectiveness of the tolerance-promoting treatment.

In some embodiments, the following protocol is followed:
obtain concentrated autologous immature dendritic cells from the patient;
UVB irradiate immature dendritic cells to change their APC function to tolerogenic (i.e., lacking expression of co-stimulatory molecules);
pulse irradiated dendritic cells with antigen to provide antigen—tolerogenic dendritic cell complexes. The antigen can be βAP 1-42 or βAP 1-40 for AD, or e-selectin for stroke;
inject UVB irradiated, antigen pulsed dendritic cell complexes into the patient.

Preferably, the UVB irradiated, antigen pulsed dendritic cell complexes are injected into a lymph node, wherein they combine with naive T cells to produce antigen-specific T regulatory cells (T_{reg}). In vivo, tolerogenic T_{reg} cells will be restimulated with antigen to produce IL-10 and TGF-B.

The injection of the T regulatory cells into the patient can be either into the patient's blood of into the CSF.

Support for the concept of providing UVB irradiation of immature dendritic cells to induce tolerance to antigens is found in the literature, wherein it has been reported that UVB irradiation of such cells induces nonproliferating regulatory type T cells. Simon, Skin Pharmacol. Appl. Skin Physiol. 2002 15:330-334.

In some embodiments, the following protocol is followed:
obtain concentrated autologous immature dendritic cells and naive T cells from the patient;
UVB irradiate immature dendritic cells to change their APC function to tolerogenic (i.e., lacking expression of co-stimulatory molecules);
pulse irradiated dendritic cells with antigen to provide antigen—tolerogenic dendritic cell complexes. The antigen can be βAP 1-42 or βAP 1-40 for AD, or e-selectin for stroke;
mix UVB irradiated, antigen pulsed dendritic cell complexes with naive T cells to produce antigen-specific T regulatory cells (T_{reg}); and
inject T regulatory cells into the patient.

In vivo, tolerogenic T_{reg} cells will be restimulated with antigen to produce IL-10 and TGF-B.

The injection of the T regulatory cells into the patient can be either into the patient's blood of into the CSF.

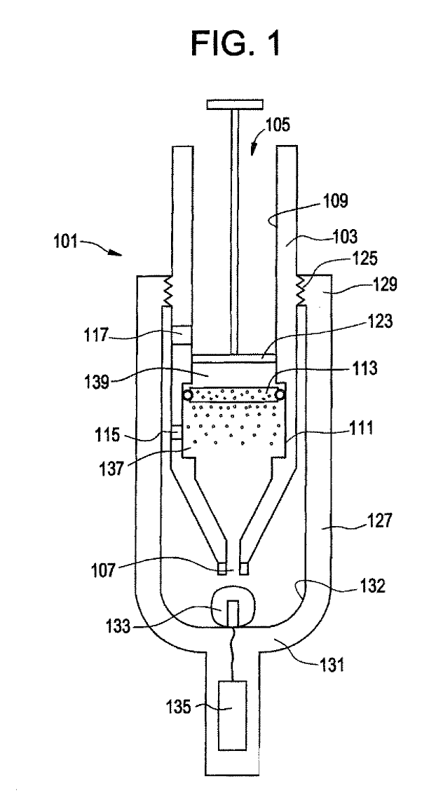

Now referring to FIG. 1, there is provided a syringe 101 adapted for tolerizing the WBCs of the present invention. This syringe is adapted to receive concentrated cells, dewater the cells, receive compounds such as βAP-1-42, receive UVB light, and finally deliver the tolerized cells to the patient.

The syringe comprises a barrel 103 having an inner wall 109, a proximal open end 105 and a distal open end 107. A recess 111 is provided in a portion of the inner wall in order to accommodate axial sliding of moveable filter 113. The syringe further has side ports 115 and 117 having gaskets 119 and 121 therein. The syringe further include a plunger having a distal plug 123, and a threaded portion 125 adapted for threadable connection to a UVB source.

The apparatus as shown further includes a UVB source 127 adapted for connection to the syringe. The purpose of the UVB source is to reliably produce an appropriate dose of UVB radiation to the WBC cells. The UVB source has a threaded end 129 adapted for threadable connection with the corresponding thread on the outer surface of the syringe. The UVB source has a closed end 131 having an inner surface 132 having a cup shape which houses a UVB light 133 connected to an energy source 135. The inner surface is preferably made of a reflective material to direct the UVB light towards the WBCs, while cup shape of the inner surface also direct the UVB light towards the WBCs

In use, the clinician adds the concentrated cells (preferably, at least PBMCs) to the chamber 137 defined by the syringe barrel and filter. Next, the βAP 1-42 is added to the chamber, optionally through port 115. Next, the UVB source is threaded onto the syringe and the UVB source is activated to irradiate the cells with an effective amount of UVB light. Next, plunger is partially withdrawn from the barrel, thereby creating a vacuum and drawings fluid from the chamber 137 into space 139. A needle is then inserted into space 139 through port 117 in order to remove the withdrawn fluid.

Next, cryoprecipitate fibrinogen and thrombin are added to the chamber through port 115 in order to begin the clotting process, which keeps the cells localized.

Lastly, the plunger is advanced so that the contents of the chamber 37 are injected into the vicinity of a lymph node.

It is further believed that analogous procedures may be carried out for the treatment of multiple sclerosis and stroke, wherein myelin basic protein (MBP) replaces BAβ 1-42 as the antigen. It has been reported in the literature that nasal adminstration of an effective amount of MBP reduces the severity of stroke and multiple sclerosis. For example, Becker, Stroke, 2003, 34, 1809-15 reports that exposing the nasal mucosa to MBP results in tolerized lymphocytes in the circulatory system. Similarly, Stohlman, J. Immunology, 1999, 163:6338-44 reports that peripheral Th2 cells that were activated by MBP antigen was able to attenuate EAE by their secretion of IL-10.

In some embodiments, the MBP is provided intranasally in an amount of between 0.2 g to 10 g for a human adult. This dose is provided between about 3 and 10 times on a quasi-daily basis. When MBP is selected as the antigen, the methods described in U.S. Pat. No. 6,068,884 (Becker), the specification of which is incorporated by reference in its entirety, may be used.

### EXAMPLE I

This prophetic example describes a typical method of the present invention.

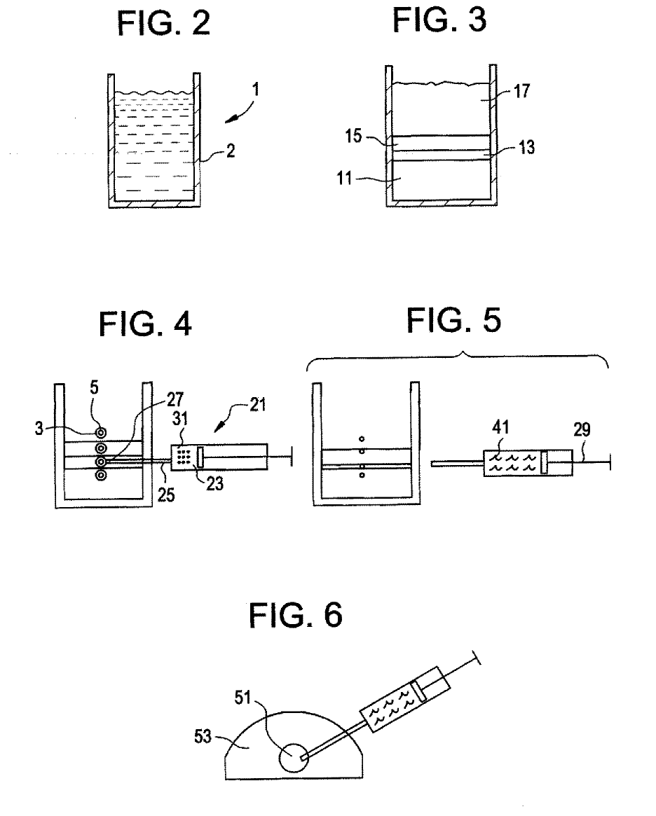

First, about 20 cc of blood is taken from the patient. Now referring to FIG. 2, the blood is placed in a centrifugation container 1 adapted for centrifugation and having a side wall 2.

Now referring to FIG. 3, the blood is centrifuged to produce centrigued blood fractions including red blood cells 11, platelets 13, buffy coat 15 and platelet poor plasma 17.

Now referring to FIG. 4, a syringe 21 having a barrel 23 containing a fluid 31 comprising BAP 1-42 and PAGE and a needle 25 is provided. The centrifugation container has a plurality of side ports 3 having puncturable gaskets 5 therein. The clinician inserts the distal end 27 of the needle through the lowest gasket in the buffy coat portion of the fractionated blood.

Now referring to FIG. 5, the clinician pulls back upon the plunger 29. The vacuum created by withdrawl of the plunger causes the buffy coat fluid to enter the barrel of the syringe, thereby reconstituting the βAP 1-42 protein and producing a βAP 1-42—rich buffy coat fluid 41.

Next, the clinician attached the UVB source and provides an effective amount of UVB light to the WBCs.

After reconstitution of the βAP 1-42 protein, the clinician then waits about 2 hours in order for the βAP 1-42 protein to interact with the monocytes in the fluid.

Next, the physician partially withdraws the plunger and dewaters the formulation.

Next, the clinician receives the syringe having the inventive composition of the present invention. This syringe has a small gauge needle, typically a 22 or 24 gauge needle. The barrel of the syringe contains the formulation of the present invention.

Finally, and now referring to FIG. 6, the clincian depresses the plunger of the syringe, thereby injecting between about 0.5 and 1 ml of the formulation comprising tolerized cells and BAP 1-42 into a region 53 in the vicinity of a lymph node 51, or into the lymph node 51 itself.

We claim:

1. A method of treating Alzheimer's Disease in a patient, comprising the steps of:
a) irradiating autologous white blood cells with UVB light to cause tolerance therein, and
b) combining the tolerized cells with βAP 1-42 to form a mixture.

2. The method of claim 1 further comprising the step of:
c) injecting the mixture into the patient in the vicinity of a lymph node.

3. The method of claim 1 wherein the white blood cells comprise monocytes.

4. The method of claim 3 wherein the monocytes are present in a concentration of at least 10⁶/cc.

5. The method of claim 1 wherein the white blood cells comprise lymphocytes.

6. The method of claim 1 wherein the white blood cells comprise dendritic cells.

7. The method of claim 1 wherein the UVB light is narrowband UVB.

8. The method of claim 1 wherein the UVB light has a maximum emission of 311-312 nm.

9. The method of claim 1 wherein the UVB light irradiates the cells with between about 0.02 J/cm² and 20 J/cm² energy.

10. The method of claim 1 wherein the βAP 1-42 is recombinant.

11. The method of claim 1 wherein the βAP 1-42 is autologous.

12. The method of claim 1 wherein the βAP 1-42 is obtained from a thyroid of the patient.

13. The method of claim 1 wherein the βAP 1-42 is obtained from blood of the patient.

14. The method of claim 1 wherein the βAP 1-42 is obtained from CSF of the patient.

15. The method of claim 1 further comprising the step of:
c) adding a metal selected from the group consisting of aluminum, copper, iron and zinc to the mixture.

16. A kit for treating AD, comprising:
a) a UVB light source, and
b) PAGE.

17. A kit for treating AD, comprising:
a) a UVB light source, and
b) βAP 1-42.

18. A kit for treating AD, comprising:
a) a metal selected from the group consisting of aluminum, copper, zinc and iron, and
b) βAP 1-42.

19. A method of treating stroke or multiple sclerosis in a patient, comprising the steps of:
a) irradiating autologous white blood cells with UVB light to cause tolerance therein, and
b) combining the tolerized cells with myelin basic protein to form a mixture.

20. A method of treating a neurodegenerative disease in a patient, comprising the steps of:
a) obtaining concentrated autologous immature dendritic cells from the patient;
b) UVB irradiating the immature dendritic cells to cause a tolerogenic state;
c) pulsing tolerogenic dendritic cells with an antigen to provide antigen—tolerogenic dendritic cell complexes;
d) injecting UVB irradiated, antigen pulsed dendritic cell complexes into the patient.

21. The method of claim 20 wherein the antigen is βAP 1-42.

22. The method of claim 20 wherein the antigen is e-selectin.

23. The method of claim 20 wherein the UVB irradiated, antigen pulsed dendritic cell complexes are injected into a lymph node.

24. A method of treating a neurodegenerative disease in a patient, comprising the steps of:
a) obtaining concentrated autologous immature dendritic cells and naive T cells from the patient;
b) UVB irradiating the immature dendritic cells to cause a tolerogenic state;
c) pulsing tolerogenic dendritic cells with an antigen to provide antigen—tolerogenic dendritic cell complexes;
d) mixing UVB irradiated, antigen pulsed dendritic cell complexes with naive autologous T cells to produce antigen-specific T regulatory cells (T_{reg}); and
e) injecting the T regulatory cells into the patient.

25. The method of claim 24 wherein the antigen is βAP 1-42.

26. The method of claim 24 wherein the antigen is e-selectin.
